(19) United States
(12) Patent Application Publication
(10) Pub. No.: US 2008/0286294 A1
(43) Pub. Date: Nov. 20, 2008

(54) Nodavirus-Vlp Immunization Composition

(75) Inventors: Richard Thiery, Biot (FR); Marine Baud, Brest (FR); Joelle Cabon, Plabennec (FR); Joelle Cozien, Ploumoguer (FR); Francois Lamour, Brest (FR); Chan-Shing Lin, Surrey (CA); Neel Krishna, Norfolk, VA (US); John E. Johnson, San Diego, CA (US); Anette Schneemann, San Diego, CA (US)

Correspondence Address: FOLEY AND LARDNER LLP;SUITE 500, 3000 K STREET NW, WASHINGTON, DC, 20007, US

(73) Assignees: AGENCE FRANCAISE DE SECURITE SANITAIRE DES ALIMENT, Maisons-Alfort (FR); THE SCRIPPS RESEARCH INSTITUTE, La Jolla, CA (US)

(21) Appl. No.: 11/596,860

(22) PCT Filed: May 19, 2005
(86) PCT No.: PCT/IB05/01863

§ 371 Date: Nov. 17, 2006

Related U.S. Application Data
(60) Provisional application No. 60/572,125, filed on May 19, 2004.

(51) Int. Cl. A61K 39/12

(52) U.S. Cl. 424/186.1; 424/204.1

(57) ABSTRACT

The present invention relates to an immunogenic composition for fish comprising nodavirus virus-like particles (VLPs) formed with nodavirus capsid protein assembly, for use as a vaccine. This composition is suitable for administration to fish via the intramuscular or intraperitoneal route, or by bath and/or via the oral route. The invention also relates to the use of such VPLs for the manufacturing of a vaccine for treating or preventing fish against a nodavirus infection. Fish farming baths and concentrates comprising the nodavirus VLP composition, as well as fish farming methods, are also encompassed in the present invention.

The present invention relates to an immunogenic composition for fish comprising nodavirus virus-like particles (VLPs) formed with nodavirus capsid protein assembly, for use as a vaccine. This composition is suitable for administration to fish via the intramuscular or intraperitoneal route, or by bath and/or via the oral route. The invention also relates to the use of such VPLs for the manufacturing of a vaccine for treating or preventing fish against a nodavirus infection. Fish farming baths and concentrates comprising the nodavirus VLP composition, as well as fish farming methods, are also encompassed in the present invention.

Betanodavirus is a recently recognized genus of family Nodaviridae which was previously known only in insects (Ball et al., 2000, in Virus taxonomy, Seventh report of the international committee on taxonomy of viruses. pp 747-755, Van Rengenmortel, M. H. V. Eds, Academic press, New York). Viruses belonging to this genus are the causative agent of viral encephalopathy and retinopathy (VER), also called viral nervous necrosis (VNN), a devastating disease of many species of marine fish cultured worldwide (Munday et al., 2002, J. Fish Dis., 25, 127-142). Affected fish commonly display neurological disorders, which are often associated with strong vacuolisation of the central nervous system and the retina.

At present, there is no treatment nor any commercial vaccine to prevent this disease in fish. The control of the disease is based upon the virus detection in contaminated animals that rely on several diagnostic methods including isolation of the causative agent and/or detection of virus component such as antigens or genome fragments. Infected animals are eliminated. Selection of putative virus-free breeders can also be performed by specific antinodavirus antibody screening of the broodstock. Strict disinfection procedures using various physical or chemical agents capable of inactivating VNN viruses are also recommended in infected farms but are difficult to apply in practice.

It is widely accepted that a vaccine capable of preventing viral nervous necrosis in fish populations would be a great improvement leading to an effective control and to the reduction of economic loss in the fish industry.

Betanodaviruses are small, spherical, non-enveloped viruses with a genome composed of two single strand RNA molecules of positive sense. The larger genomic segment, RNA1 (3.1 kb), encodes the RNA-dependent RNA polymerase (Chi & Lin, 2001, J Fish Dis, 24, 3-14, Nagai & Nishizawa, 1999, J Gen Virol, 80, 3019-3022, Tan et al., 2001, J Gen Virol, 82, 647-653); whereas the coat protein is encoded by RNA 2 (1.4 kb) (Delsert et al., 1997, Arch Virol, 142, 2359-2371, Nishizawa et al., 1995, J Gen Virol, 76, 1563-1569.). Betanodaviruses are classified in different groups, depending on these RNA genomic fragments, including among others SJNNV (striped jack nervous necrosis virus), TPNNV (tiger puffer nervous necrosis virus), BFNNV (barfin flounder nervous necrosis virus), RGNNV (red grouper nervous necrosis virus). MGNNV (malabaricus grouper nervous necrosis virus), DGNNV (dragon grouper nervous necrosis virus) and dicentrarchus labrax encephalitis viruses (isolates V26 also called SB2, and Y235 also called SB1) belong to the RGNNV group (Thiéry et al, 2004, J Gen Virol. 85, 3079-3087). SB1 and SB2 strains are classified in different subtypes within the RGNNV group.

Nodavirus VLPs (virus-like particles) have been obtained using RNA2 encoding the nodaviral capsid protein (Lin et al., Virology. 2001, 290, 50-58): the recombinant capsid protein spontaneously assembles in VLPs, that closely resemble to the native virion on a morphological basis, but that do not contain any infectious genetic material. The present invention demonstrates for the first time that nodavirus VLPs originating from different nodavirus strains are immunogenic and can be used as an immunogenic composition to prevent fish from viral nervous necrosis. Moreover, the present invention demonstrates that a protection can be obtained further to the administration of such an immunogenic composition via the intra-muscular or intra-peritoneal route, or by bath and/or oral exposure.

### DESCRIPTION

Thus, in a first aspect, the invention is aimed at a immunogenic composition for fish, wherein it comprises nodavirus virus-like particles (VLPs) which are formed with nodavirus capsid protein assembly. This composition is especially suited for use as a vaccine.

The composition of the invention is also termed herein indifferently “immunization composition”, “vaccine” or “vaccine composition”.

The VLPs which are formed with nodavirus capsid protein assembly can be produced by any suitable method known in the art: the nucleic acid sequence encoding a nodavirus capsid protein may be cloned using known appropriate primers and reverse-transcriptase polymerase chain reaction (RT-PCR) followed by ligation in E. coli and amplification to obtain cDNA clones, using total viral RNA as template. The total viral RNA may be isolated from brains of infected fish. After DNA cloning, the cDNA may be introduced in an expression vector for expression in a suitable transformed host. Alternatively, the nodavirus capsid protein could be directly expressed in vitro by means of a cell free system. Such a system could include for example direct expression of the nodavirus capsid protein sequence by mean of a coupled transcription/translation system, using for example T7 promoter regulatory sequences and T7 polymerase. The nucleic acid sequence encoding the nodavirus capsid protein and the corresponding assembled VLPs, which are now well known by the skilled person, may be from any nervous necrosis virus origin, provided that an immunogenic response is obtained in fish using such VLPs.

The choice of expression control sequences and expression vectors will depend upon the choice of the host cells. A wide variety of expression host/vector combinations may be employed. For example, useful expression vectors for bacterial hosts include known bacterial plasmids, such as plasmids from E. coli (for example pET derivatives). Insect cells supporting recombinant baculovirus replication such as Spodoptera Frugiperda (Sf9, Sf21, . . . ) cells or Tricoplusia ni (T. ni) cells may also be used for the obtention of the nodavirus VLPs. The Sf21 cells are adapted to serum-free suspension culture for transient or stable expression of recombinant proteins. These cells may be obtained for example at Invitrogen/Product Cat. No. Sf21 Cells, SFM Adapted 3 ml 11497-013. T. ni cells may be obtained for example at Orbigen Cat No CEL-10005. Advantageously, such T. ni cells provide a larger scale synthesis of VLPs than Sf21 cells.

Insect constitutive vectors available for expression of proteins in cultured insect cells include the pAc series (Smith et al., (1983) Mol. Cell. Biol. 3:2156-2165) and the pVL series (Lucklow, V. A., and Summers, M. D., (1989) Virology 170:31-39).

Methods for DNA cloning and expression in host cells using appropriate vectors are well known in the art. See, for example, Sambrook et al., Molecular Cloning: A Laboratory Manual, Cold Spring Harbor Laboratory, Cold Spring Harbor, N.Y. (1989) and Ausubel et al., Current Protocols in Molecular Biology, J. Wiley & Sons, NY (1992).

Another suitable expression system for nodavirus capsid protein is a yeast expression vector. Examples of vectors for expression in yeast S. cerevisiae include pYepSec1 (Baldari, et al., (1987) EMBO J. 6:229-234), pMFa (Kujan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Further details and protocols for preparing the recombinant baculovirus, the purification of VLPs, are exemplified hereinafter.

A preferred embodiment is a composition as defined above, wherein the VLPs comprise at least one nodavirus capsid protein selected from the group comprising:

a) sequences SEQ ID No 5, SEQ ID No 6, SEQ ID No 7 and SEQ ID No 8; and
b) sequences having at least 70%, 80%, 90%, 95% or 99% of identity with the sequences as defined in (a).

Another preferred embodiment is the nodavirus VLP composition of the present invention, wherein the nodavirus capsid protein is encoded by a nucleic acid selected from the group comprising:

a) sequences SEQ ID No 1, SEQ ID No 2, SEQ ID No 3 and SEQ ID No 4; and
b) sequences having at least 70%, 80%, 90%, 95% or 99% of identity with the sequences as defined in (a).

Since natural nodavirus capsids are composed of a unique coat protein (encoded by RNA2), similarly the nodavirus VLPs of the present invention, which are advantageously obtained using recombinant expression systems, are preferably composed of a unique nodavirus capsid protein. However, nodavirus VLPs composed of different nodavirus coat proteins, called herein “chimeric VLPs”, are also encompassed in the present invention. Thus, in another embodiment of the present invention, the VLPs comprise at least two or three different nodavirus capsid proteins. Such VLPs may be obtained from the expression of recombinant vectors encoding nodavirus capsid proteins from different nervous necrosis virus in a host cell culture. Similarly, such VLPs may be obtained from the expression of a recombinant vector encoding two different nodavirus capsid proteins in a host cell culture.

By percentage of identity between two nucleic acid or amino acid sequences in the present invention, it is meant a percentage of identical nucleotides or amino acid residues between the two sequences to compare, obtained after the best alignment; this percentage is purely statistical, and the differences between the two sequences are randomly distributed and all along their length. The best alignment or optimal alignment is the alignment corresponding to the highest percentage of identity between the two sequences to compare, which is calculated such as herein after. The sequence comparisons between two nucleic acid or amino acid sequences are usually performed by comparing these sequences after their optimal alignment, said comparison being performed for one segment or for one “comparison window”, to identify and compare local regions of sequence similarity. The optimal alignment of sequences for the comparison can be performed manually or by means of the algorithm of local homology of Smith and Waterman (1981) (Ad. App. Math. 2:482), by means of the algorithm of local homology of Neddleman and Wunsch (1970) (J. Mol. Biol. 48:443), by means of the similarity research method of Pearson and Lipman (1988) (Proc. Natl. Acad. Sci. USA 85:2444), by means of computer softwares using these algorithms (GAP, BESTFIT, FASTA and TFASTA in the Wisconsin Genetics Software Package, Genetics Computer Group, 575 Science Dr., Madison, Wis.).

The percentage of identity between two nucleic acid or amino acid sequences is determined by comparing these two aligned sequences in an optimal manner with a “comparison window” in which the region of the nucleic acid or amino acid sequence to compare may comprise additions or deletions with regard the sequence of reference for an optimal alignment between these two sequences. The percentage of identity is calculated by determining the number of positions for which the nucleotide or the amino acid residue is identical between the two sequences, by dividing this number of identical positions by the total number of positions in the “comparison window” and by multiplying the result obtained by 100, to obtain the percentage of identity between these two sequences.

Still another preferred embodiment is the nodavirus VLP composition of the present invention, wherein the VLPs are obtained by the following process:
infecting host cells with a recombinant vector capable of expressing the nodavirus capsid protein,
obtaining a host cell lysate comprising the nodavirus VLPs, and
optionally extracting and purifying the VLPs assembled from the host cell lysate.

The extraction and purification of VLPs from a host cell lysate are well known by the man skilled in the art and may comprise, for example, sedimentation such as centrifugation.

In one particular aspect, the immunogenic composition of the invention comprises a lysate of infected host cells with a recombinant vector capable of expressing the nodavirus capsid protein.

Preferably, the recombinant vector is a recombinant baculovirus and the host cells are insect cells. More preferably, the insect cells are Sf21 cells or T. ni cells.

Most preferably, the nodavirus capsid protein is encoded by the nucleic acid as defined above.

In a further preferred embodiment, the composition of the present invention comprises a mixture of VLPs with at least two VLPs, preferably at least three, four or five VLPs, each VLP comprising a nodavirus capsid protein which is different from that of the other VLP. Such a mixture may also comprise different chimeric VLPs.

Still more preferably, the nodavirus VLP composition of the present invention further comprises a pharmaceutically acceptable adjuvant.

Conventional and pharmaceutically acceptable adjuvants, such as for example but without any limitation, mineral gels, e.g., aluminum hydroxide; surface active substances such as lysolecithin, pluronic polyols; polyanions; peptides; oil emulsions, incomplete Freund's adjuvant maybe used.

In a particularly preferred embodiment, the nodavirus VLP composition of the present invention is suitable for administration to fish.

The expression “suitable for administration” means that VLPs which are to be administered as vaccines can be formulated according to conventional methods known from the skilled person for such administration to fish to be protected, and can be mixed with conventional and pharmaceutically acceptable adjuvants. Such an administration includes the intramuscular or the intraperitoneal route, as well as by bath and/or the oral route.

It will be understood that the composition of the invention may comprise purified VLPs as defined above or lysate of host cells infected with a recombinant vector capable of expressing the nodavirus capsid protein.

The invention also relates to a food composition for fish comprising a suitable amount of nodavirus virus-like particle (VLP), wherein said VLPs are formed with nodavirus capsid protein assembly. Such composition is used as a vaccine. Preferably, the food composition comprises VLPs which comprise at least one nodavirus capsid protein selected from the group consisting of:

a) sequences SEQ ID No 5, SEQ ID No 6, SEQ ID No 7 and SEQ ID No 8; and
b) sequences having at least 80%, 90%, 95% or 99% of identity with the sequences as defined in (a).

Preferably, the fish is from Dicentrarchus labrax species, Epinephelus sp. or any fish species susceptible to nodavirus infection. An unlimited list of fish species is available in Munday et al., 2002, J. Fish Dis., 25, 127-142. Accordingly, the invention can be practiced in fish species raised for food as well as ornamental fishes in aquarium such as marine tropical fishes and other ornamental fishes including fresh water fishes such as guppies.

Advantageously, the nodavirus VLP composition of the present invention is suitable for administration via the intramuscular or the intraperitoneal route.

Preferably, the nodavirus VLP composition of the present invention comprises between 0.5 μg and 200 μg of VLPs for 100 g of fish. More preferably, the nodavirus VLP composition comprises between 1 μg and 20 μg of VLPs for 100 g of fish. Still more preferably, the nodavirus VLP composition comprises between 1 and 5 μg of VLPs for 100 g of fish.

Alternately, the nodavirus VLP composition of the present invention is suitable for administration by bath and/or via the oral route.

Preferably, the nodavirus VLP composition comprises between 0.5 μg and 100 μg of VLPs for 100 g of fish. More preferably, the nodavirus VLP composition comprises between 500 μg and 150 mg of VLPs for 100 g of fish. Still more preferably, the nodavirus VLP composition comprises between 1 mg and 100 mg of VLPs for 100 g of fish.

In a second aspect, the invention relates to the use of the nodavirus VLP as defined above for the manufacturing of a vaccine for treating or preventing fish against a nodavirus infection.

Advantageously, the invention relates to the use of the nodavirus VLP as defined above for the manufacturing of an immunogenic composition or medicament for preventing or treating viral encephalopathy, retinopathy or viral nervous necrosis in fish.

In another preferred embodiment, fish are raised in a fish farming, and are preferably at the larval and juvenile stage of development or broodstock fish.

In a third aspect, the invention relates a fish farming bath comprising the nodavirus VLP composition of the present invention, wherein said nodavirus VLP composition comprises between 0.5 μg and 200 mg of VLPs for 100 g of fish.

In a fourth aspect, the invention relates to a concentrate of the nodavirus VLP composition as defined in the present invention which is suitable for treating or preventing fish in farming baths from nodavirus infection.

In a fifth aspect, the invention relates to a method of treatment or prevention of nodavirus infection comprising introducing fish in a treatment bath comprising an appropriate amount of the nodavirus VLP composition of the present invention or of the concentrate of the present invention, during an appropriate time to allow stimulation of the fish immune system.

By appropriate amount of the nodavirus VLP composition it is meant herein an amount of the nodavirus VLP composition which is sufficient to obtain an immunogenic response (detection of anti-nodavirus antibodies in vaccinated fish). Preferably, said nodavirus VLP composition comprises between 0.5 μg and 200 mg of VLPs for 100 g of fish.

In a sixth aspect, the invention relates to a method for preventing or treating nodavirus infection in fish comprising administering the nodavirus VLP composition of the present invention.

The particular embodiments which are described herein relative to the first claimed subject matter are also suitable for the other aspects (second to sixth) of the present invention.

The invention is further embodied in the following examples and figures. Supplying of these examples and figures is for illustrating the present invention and do not limit the scope of the protection.

### LEGENDS OF THE FIGURES

FIG. 1: Average cumulated mortality after nodavirus challenge. Fish were vaccinated with MGNNV-VLPs (approx. 20 μg or 100 μg per fish) by intramuscular injection. Twenty seven (27) days post-vaccination fish were challenged with strain W80 (10⁵ TCID₅₀/fish). Fish mortality was recorded daily. The percentage of average cumulated mortality for each group of fish (20 μg or 100 μg) is plotted against the number of days after challenge. Unvaccinated control fish received 100 μl of PBS.

FIG. 2: Anti-nodavirus antibodies—ELISA dilution 1/8192. Fish were vaccinated with MGNNV-VLPs (approx. 20 μg or 100 μg per fish) by intramuscular injection or received 100 μl of PBS. Two-times serial dilutions of plasma from 5 individual fish per aquaria were assayed for the presence of antinodavirus antibodies by using a sandwich ELISA method. Detection was performed by colorimetic reading at OD 492 nm. Average OD readings at plasma dilution 1/8192 for each group of fish is indicated (A1, A2, A3: fish vaccinated with 20 μg MGNNV-VLPs; B1, B2, B3: fish vaccinated with 100 μg MGNNV-VLPs; A5, B5: fish treated with 100 μl of PBS.

FIG. 3: Average cumulated mortality after nodavirus challenge. Fish were vaccinated with SB2-VLPs at different doses (approx. 5 μg to 20 μg per fish) by intramuscular injection. Twenty nine (29) days post-vaccination fish were challenged with nodavirus strain W80 (10⁵ TCID₅₀/fish). Fish mortality was recorded daily. The percentage of average cumulated mortality for each group of fish at day 29 post nodaviral challenge is indicated.

FIG. 4: ELISA results. Fish were vaccinated with SB2-VLPs (approx. 5 μg to 20 μg per fish) by intramuscular injection or received 100 μl of PBS. Two-times serial dilutions of plasma from 5 individual fish per aquaria were assayed for the presence of antinodavirus antibodies by using a sandwich ELISA method. Detection was performed by colorimetic reading at OD 492 nm. Average OD readings at plasma dilution 1/8192 for each group of fish is indicated.

FIG. 5: Titration of plasmatic antinodavirus antibodies by ELISA. Blood samples were taken from 5 fish from each aquaria 19 days after the vaccination. Two-times serial dilutions of plasma from individual fish were assayed for the presence of antinodavirus antibodies by using a sandwich ELISA method. Detection was performed by colorimetic reading at OD 492 nm. Average OD readings for each plasma dilution plus the standard deviation from each fish group was plotted against the plasma dilution on a semi-logarithmic scale.

FIG. 6: Average cumulated mortality after nodavirus challenge. Thirty days before challenge, vaccine preparations containing purified or partially purified SB2-VLPs were used to treat fish by bath exposure or by intraperitonel injection (approx. 5 μg or 50 μg per fish). Negative controls included fish treatments using partially purified fractions of uninfected Tni cell lysates, either by bath exposure or by intraperitoneal injection, or PBS buffer by intraperitoneal injection. Fish were challenged with strain W80 (9×10⁶ TCID₅₀/per fish). Fish mortality were recorded daily. The percentage of average cumulated mortality for each group of fish is plotted against the number of days after challenge.

FIG. 7: Titration of plasmatic antinodavirus antibodies by ELISA. Blood samples were taken from 5 fish from each aquaria 28 days after the vaccination. Two-times serial dilutions of plasma from individual fish were assayed for the presence of antinodavirus antibodies by using a sandwich ELISA method. Detection was performed by colorimetic reading at OD 492 μm. Average OD readings for each plasma dilution plus the standard deviation from each fish group was plotted against the plasma dilution on a semi-logarithmic scale.

### EXAMPLES

### Example 1

### Production of MGNNV-VLPs

### Virus Isolation

Brains from infected Epinephelus malabaricus, stored frozen at −70° C., were homogenized in cold (4° C.) 10 mM Tris (pH 8) followed by filtration of the homogenate through a 0.45-μm filter. Juvenile fish (1 in.) were injected with 400 μl of the homogenate to amplify the virus. For purification, 200 μl virus suspension was centrifuged through a discontinuous 6-ml 20-35-50% sucrose gradient in 10 mM Tris (pH 8) at 140,000 g for 1 h (Nagai and Nishizawa, 1999, J Gen Virol, 80, 3019-3022). The virus was collected from the center of the tube at the end of centrifugation.

### Isolation of Total Viral RNA

To isolate total RNA from purified virus particles, the virus suspension was frozen in liquid nitrogen, crushed to powder, and resuspended in 2×LETS buffer (0.2 M LiCl, 20 mM EDTA, 2% SDS, 20 mM Tris, pH 7.8). Total RNA was then extracted with acidic (pH 4) phenol:chloroform (5:1) (Ambros, 1989, Cell, 57, 49-57) and precipitated with ethanol in the presence of 0.2 M LiCl.

cDNA Cloning of Viral RNA2

Reverse-transcriptase-polymerase chain reaction (RT-PCR) was employed to obtain cDNA clones for viral RNA2 using total viral RNA as template. Ready-To-Go RT-PCR beads (Amersham Pharmacia Biotech) were mixed with 30-50 pg RNA template, 1 μg of primers NI (5′-CGCTTTGCAAGTCAAAATGGT-3′-SEQ ID No 15) and N2 (5′-ACCA-CATGGCGGTGGTGCTCA-3′-SEQ ID No 16), and 45 μl of H₂O. The sequence of primers N1 and N2 was based on the 5′ and 3′ end sequences of RNA2 of the fish nodavirus SJNNV (striped jack nervous necrosis virus). RT-PCR conditions were as follows: reverse transcription of viral RNA for 40 min at 42° C., denaturation of RNA-DNA hybrid for 5 min at 95° C., and 30 cycles of DNA amplification (denaturation at 94° C. for 120 s; annealing at 59° C. for 120 s; extension at 72° C. for 90 s). DNA products were extended in a final step at 72° C. for 10 min and purified for direct sequencing and insertion into a sequencing vector. For insertion into a sequencing vector, PCR products were reamplified with primers NW1 (5′-CGCTTTGGAATTCAAAATGGT-3′-SEQ ID No 17) and NW2 (5′-TTTATCTAGATGGCGGTG-3′-SEQ ID No 18), which incorporated an EcoR1 and an XbaI restriction site, respectively. PCR was performed using the same conditions as described above except that annealing was performed at 47° C. for 120 s. The products were digested with EcoRI and XbaI and ligated into either pTTQ18 or pUC19, which had been digested with the same enzymes. The plasmid containing the cDNA of MGNNV (malabaricus grouper nervous necrosis virus) RNA2, the virus isolated from E. malabaricus, was called pTA.

### Sequence Analysis

Plasmid DNA and PCR products containing viral cDNAs were purified using Qiagen plasmid miniprep spin columns. Sequence analysis was performed on an ALFexpress II DNA Analysis System (Amersham Pharmacia Biotech) using the Autoread Sequencing kit with Cy5-labeled primers. Each sample was sequenced at least three times for increased accuracy. The sequence of RNA2 of MGNNV has been submitted to GenBank and can be retrieved using Accession No. AF245003 (SEQ ID No 4).

### Insect Cell Culture

Spodoptera frugiperda cells (line IPLB-Sf21) were grown at 27° C. in TC100 medium supplemented with 0.35 g of NaHCO₃ per liter, 2.6 g of tryptose broth per liter, and 10% heat-inactivated fetal bovine serum. Cultures were maintained as monolayers in screw-capped plastic flasks or as suspensions in 1-L spinner flasks (Bellco, Vineland, N.J.). Further details are given is the following examples.

### Construction of Recombinant Baculovirus Containing the Gene for the MGNNV Coat Protein

A recombinant baculovirus containing the gene for the MGNNV coat protein was generated using the BacPAK baculovius expression system kit (Clontech). To this end, the cDNA of MGNNV RNA2 was released from plasmid pTA by digestion with EcoRI and XbaI and inserted into pBacPAK9 digested with the same enzymes. The resulting plasmid, pB9M, was mixed with Bsu361-linearized BacPAK6 viral DNA and transfected into Sf21 cells following protocols provided by the manufacturer. Three days after transfection, cell supernatants were harvested and putative recombinant viruses were isolated by plaquing the supernatants once on Sf21 monolayers. Individual plaque isolates were amplified following confirmation of the presence and expression of the MGNNV coat protein gene. The recombinant virus selected for all further experiments was called BV-B9M.

### Synthesis and Purification of MGNNV VLPs

Monolayers consisting of 2-4×10⁶ Sf21 cells per 100-mm tissue culture dish were infected with recombinant baculovirus at a multiplicity of 0.5-2 PFU per cell. The virus was added in a total volume of 1 ml and allowed to attach to the cells at room temperature with gentle rocking. After 1 h, 5 ml of growth medium was added to the cells and incubation was continued at 27° C. for approximately 3 days. At 60-72 h postinfection, infected cells were harvested, pelleted at 3800 g, resuspended in 10 mM Tris buffer (pH 8), and stored frozen at −20° C. until further analysis. Cells were thawed in a 37° C. waterbath for 10 min either in the presence or in the absence of RNase A at a final concentration of 10 μg/ml. Nonidet-P40 was then added to a final concentration of 0.5% (v/v) to lyse the cells and the sample was incubated at 4° C. overnight. Cell debris was pelleted in a Beckman JA17 rotor at 10,000 rpm (13,800 g) for 10 min at 4° C. and VLPs in the supernatant were pelleted through a 4-ml 20% (wt/wt) sucrose cushion in 10 mM Tris (pH 8) at 28,000 rpm (141,000 g) in an SW28 rotor for 5.5 h at 11° C. The pellet was resuspended in 10 mM Tris (pH 8) and layered on an 11-ml 10-40% (wt/wt) sucrose gradient in the same buffer. VLPs were sedimented at 40,000 rpm (274,000 g) in an SW41 rotor for 1.5 h at 11° C. The gradient was fractionated on an ISCO gradient fractionator at 0.75 ml/min and 0.5 min per fraction. Further details are given in the following examples.

### Large Scale Synthesis and Purification of MGNNV VLPs.

A 1 L T. ni cell culture (Cat No CEL-10005) at a density of approximately 2×10⁶ cells/ml was infected with 30 ml of PASS3 recombinant baculovirus stock and incubated at 27° C. for three days. Cells were then pelleted and the supernatant discarded. The cells were resuspended in 200 ml 10 mM Tris pH 8 and lysed with NP40 (0.5% v/v final concentration). The cell debris was pelleted and the supernatant was transferred to ultracentrifuge tubes. Samples were underlayed with 20% (wt/wt) sucrose and VLPs were pelleted by centrifugation in a Ti50.2 rotor at 45,000 rpm (245,000×g) at 11° C. for 2.5 hours. The tubes were drained and each pellet was resuspended in 0.5 ml 10 mM Tris pH 8. The resuspended pellets were combined and RNaseA was added to a final concentration of 5 μg/ml and MgCl₂ to 5 mM final concentration. The sample was incubated at room temperature for 10 min and insoluble debris was removed by low speed centrifugation. The sample was subsequently layered on 10-40% (wt/wt) sucrose gradients and centrifuged at 141,000×g for 3 hours at 11° C. The gradients were fractionated and 2-3 μL of each fraction were analyzed on a protein gel. Fractions containing MGNNV VLPs were pooled and dialysed against 10 mM Tris pH 8, 10 mM NaCl. RNaseA (1 μg/ml) and MgCl₂ (5 mM) were added and the sample incubated at room temperature for 30 min. A precipitate that formed during the incubation was removed by low speed centrifugation and the clarified sample was centrifuged in 32% (wt/wt) CsCl overnight at 11° C. The resulting gradient was fractionated and fractions containing the VLPs were pooled. The pooled sample was dialyzed against 10 mM Tris pH 8 and concentrated. The VLPs concentration was estimated by comparison of protein bands with that of known amount of an insect nodavirus after polyacrylamide gel elecrophoresis. Further details are given in the following examples.

### Example 2

### Production of SB1-VLPs and SB2-VLPs

cDNA fragments encoding the capsid protein of the so-called SB1 or SB2 strains of sea bass nodavirus were obtained by RT-PCR using total RNA extracted form diseased larvae or juveniles from seabass Dicentrarchus labrax reared in France. Primers used for amplification were derived from SEQ ID No 2 (GeneBank Accession Number U39876 or SEQ ID No 3 (GeneBank Accession Number AJ698105) (strain SB1 or Y235) and from SEQ ID No 1 (GeneBank Accession Number AJ698093 (strain SB2 or V26) and both strains are available at Afssa-site de Brest, France. The DNA fragment were cloned into bacterial plasmids and propagated in E. coli according to standard protocols. The resulting plasmids were designated pSB1 and pSB2 respectively.

Construction of pBacPAK9 Transfer vectors Containing the Coding Sequence of SB1 and SB2 Coat Proteins

pSB1 and pSB2 were used as templates to amplify the coding sequence of the coat proteins of SB1 and SB2 by PCR.

The following primers were used for pSB1:

| | |
|---|---|
| SB1 N-term | |
| 5′ ACCAGATCTATGGTACGCAAGGGTGAG 3′ | (SEQ ID No 11) |
| SB1 C-term | |
| 5′ TAAGCGGCCGCTTAGTTTCCCGCATCGAC 3′ | (SEQ ID No 12) |

The following primers were used for pSB2:

| | |
|---|---|
| SB2 N-term | |
| 5′ ACCAGATCTATGGTACGCAAAGGTGAT 3′ | (SEQ ID No 9) |
| SB2 C-term | |
| 5′ TAAGCGGCCGCTTAGTTTTCCGAGTCAAC 3′ | (SEQ ID No 10) |

Both N terminal primers contained a BglII site and both C terminal primers contained a NotI site.

| | | |
|---|---|---|
| PCR: | SB1 | SB2 |

| SB1 N-term (100 ng/μl) | 2.5 | μl | — | |
|---|---|---|---|---|
| SB1 C-term (100 ng/μl) | 2.5 | μl | — | |
| SB2 N-term (100 ng/μl) | — | | 2.5 | μl |
| SB2 C-term (100 ng/μl) | — | | 2.5 | μl |
| 10× Pfu buffer | 10 | μl | 10 | μl |
| DMSO | 5 | μl | 5 | μl |
| 1.25 mM dNTPs | 16 | μl | 16 | μl |
| pSB1 | 1 | μl | — | |
| pSB2 | — | | 1 | μl |
| Pfu turbo pol (Stratagene, 2.5 units/μl) | 1 | μl | 1 | μl |
| H₂O | 62 | μl | 62 | μl |

PCR conditions:

| | | | |
|---|---|---|---|
| 95° C. 5 sec | 60° C. 15 sec. | 72° C. 1 min. | 10 cycles |
| 95° C. 5 sec. | 55° C. 15 sec. | 72° C. 1 min. | 10 cycles |
| 95° C. 5 sec. | 50° C. 15 sec. | 72° C. 1 min. | 10 cycles |
| 72° C. 10 min. | | | |
| soak at 4° C. | | | |

The PCR reactions were loaded on a 1% agarose gel in Tris-acetate EDTA (TAE) buffer and electrophoresed at 100 V for about 45 min. The amplified DNA in each reaction was excised from the gel and purified using the QIAEX II Gel extraction kit (Qiagen).

The purified PCR products were digested with BglII and NotI restriction enzymes overnight at 37° C. In parallel, the baculovirus transfer vector pBacPAK9 (Clontech) was digested with BglII and NotI.

The digested DNAs were purified again using the QIAEX II Gel extraction kit (Qiagen).

The digested SB1 and SB2 PCR products were ligated into the BglII and NotI site of pBacPAK9. Following ligation, competent E. coli (DH5α) cells were transformed with an aliquot of the ligation reaction and plated on LB agar containing ampicillin (100 μg/ml).

Colonies were screened for the presence of pBacPAK9 containing SB1 or SB2 insert using primers Bac1 and Bac2 which anneal within the pBacPAK9 vector, 5′ and 3′ to the inserted DNA.

| | |
|---|---|
| Sequence of Bac1 | |
| 5′ ACCATCTCGCAAATAAATAAG 3′ | (SEQ ID No 13) |
| Sequence of Bac2 | |
| 5′ ACAACGCACAGAATCTAGCG 3′ | (SEQ ID No 14) |

PCR:

| | | |
|---|---|---|
| Bac1 (342 ng/μl) | 0.8 | μl |
| Bac2 (392 ng/μl) | 0.7 | μl |
| 10× Taq pol buffer | 5 | μl |
| 1.25 mM dNTPs | 8 | μl |
| Taq Pol (Gibco, 5 units/μl) | 0.5 | μl |
| Bacteria from colony | | |
| H₂O | 35 | μl |

PCR conditions:

| 15 min. 95° C. | |
|---|---|
| 5 min. 55° C. | |
| 20 cycles: | 2 min 72° C. |
| | 1.5 min. 95° C. |
| | 1 min. 55° C. |
| soak at 4° C. | |

Bacteria from colonies giving positive signal in PCR were amplified in a 4 ml culture and DNA was purified using Wizard Plus Miniprep Kit (Promega).

### Synthesis of Recombinant Baculoviruses Expressing SB1 and SB2 Coat Protein.

Purified transfer vectors pBacPAK9/SB1 and pBacPAK9/SB2 were mixed with Bsu36I-digested BacPAK6 viral DNA (Clontech) and transfected into Sf21 cells for homologous recombination. Specifically, the following components were mixed in a polystyrene tube:

| | | |
|---|---|---|
| pBacPAK9/SB1 (209 ng/μl) | 1.7 | μl |
| Bsu36I-digested BacPAK6 viral DNA (Clontech) | 5 | μl |
| Bacfectin (Clontech) | 4 | μl |
| H₂O | 89.3 | μl |
| and | | |
| pBacPAK9/SB2 (173 ng/μl) | 2.9 | μl |
| Bsu36I-digested BacPAK6 viral DNA (Clontech) | 5 | μl |
| Bacfectin (Clontech) | 4 | μl |
| H₂O | 88.1 | μl |

The mixtures were incubated at room temperature for 15 min. and then added dropwise to a monolayer of 1×10⁶ Sf21 cells in a 35 mm tissue culture dish containing 1.5 ml TC100 medium lacking serum. The plate was incubated at 27° C. for 5 hours followed by addition of 1.5 ml TC100 medium containing 10% fetal bovine serum (complete TC100 medium). Incubation was continued at 27° C. for three days. At this point, the medium was removed from the cells and transferred to a sterile 15 ml conical plastic tube. The tube was labeled “transfection supernatant” and stored at 4° C.

Serial 10-fold dilutions of the transfection supernatant were prepared in complete TC100 medium. These dilutions were used in a plaque assay on Sf21 cells to isolate individual recombinant baculovirus clones encoding the SB1 and SB2 coat protein. Virus from 9-10 plaques was picked for each construct. Specifically, the narrow end of a Pasteur pipet was used to stab into the agar over the center of a plaque, the agar containing the virus was aspirated and transferred into a sterile plastic tube containing 1 ml of complete TC100.

Five plaque-purified virus recombinants were used for amplification and diagnostic tests regarding expression of SB1 and SB2 coat protein and assembly into virus-like particles (VLPs). To this end, 2.5×10⁶ Sf21 cells were plated in a 100 mm tissue culture dish and infected with the entire 1 ml volume containing virus from a plaque pick. After 1 hr incubation at room temperature, 5 ml of complete TC100 were added and the cells were incubated at 27° C. until extensive cytopathic effect (cpe) was visible (6 days). At this point, the supernatant was harvested and stored as PASS 1 at 4° C. The infected cells were pelleted, resuspended in 1 ml PBS and lysed with Nonidet P40 (NP40) (0.5% v/v final concentration). The lysate was incubated on ice for 10 min. Cell debris was then removed by centrifugation in a microcentrifuge. The supernatant was transferred to SW50.1 ultracentrifuge tubes (Beckman) and underlayed with 0.5 ml of 20% (wt/wt) sucrose in 10 mM Tris pH8. The tubes were filled to the top with PBS and centrifuged at 45,000 rpm (243,000×g) for 45 min, in an SW50.1 rotor (Beckman). After the run, the tubes were drained and the pellets, containing putative VLPs, were resuspended in 50 μl PBS. Five (5) μl were analyzed on a 12% Laemmli SDS polyacrylamide gel to determine whether SB1/SB2 coat protein was present. PASS1 of two virus recombinants judged positive by protein gel electrophoresis was amplified to yield PASS2.

PASS 2 was generated as follows: 15×10⁶ Sf21 cells in 15 ml complete TC100 were infected with 250 μl of virus from PASS1. Cells were incubated at 27° C. for one hour following addition of another 15 ml of complete TC100. Incubation at 27° C. was continued until extensive cpe was visible (5-6 days). Supernatants from the infected culture were harvested and stored as PASS2 at 4° C. PASS 3 was generated in the same manner, using 250 μl of PASS 2 for infection.

### Large Scale Synthesis and Purification of SB1 and SB2 VLPs

For large scale synthesis and purification of SB1 and SB2 VLPs we used Tricoplusia ni (T. ni) cells propagated in serum-free ExCell405 medium (JRH Biosciences). One liter of T. ni cells at a density of 2×10⁶ cells/ml was infected with 30 ml of PASS3 virus stock and incubated at 27° C. on a shaker platform (100 rpm) for four to five days.

Cell were then collected by low speed centrifugation and the supernatant discarded. The cells were resuspended in 200 ml 50 mM Hepes, 10 mM EDTA pH 7.4 and lysed by addition of NP-40 to a final concentration of 0.5% (v/v). The lysate was kept on ice for 10 min., followed by pelleting of cell debris at approximately 14,000-15,000×g for 15 min. at 4° C. The supernatants were transferred to ultracentrifuge tubes and underlayed with 30% (wt/wt) sucrose in 50 mM Hepes pH 7.4, 10 mM EDTA. VLPs were pelleted at 244,000×g for 2.5 hours at 11° C. The tubes were drained and the pellets resuspended in 50 mM Hepes pH 7.4, 10 mM EDTA. The resuspended pellets were layered on continuous 10-40% (wt/wt) sucrose gradients in 50 mM Hepes pH 7.4, 10 mM EDTA and centrifuged in an SW28 rotor at 141,000×g for 3 hours at 11° C. The gradients were fractionated and fractions containing the VLPs (as determined by protein gel electrophoresis) were pooled. The pooled fractions were dialyzed against 50 mM Hepes, pH 7.4 to remove the sucrose. The dialyzed sample was then subjected to centrifugation in CsCl using a homogeneous concentration of 32% (wt/wt). The samples were centrifuged overnight in a SW28 rotor at 112,000×g at 11° C. The gradient was fractionated and fractions containing the VLPs were pooled. In the final step, CsCl was removed by dialysis against 50 mM Hepes pH7.

Alternative purification procedure: If T. ni cells were already lysed 4-5 days after infection with recombinant baculovirus, the first few steps were as follows: NP-40 was added to the entire 1 L culture to a final concentration of 0.5% (v/v). The culture was kept on ice for 15 min. Cell debris was then removed by low speed dentrifugation. The VLPs in the supernatant (approx. 1 L) were precipitated by addition of polyethyleneglycol 8000 (PEG 8000) to a final concentration of 8% (wt/v) and NaCl to a final concentration of 0.2 M. The mixture was stirred at 4° C. for 1 hour and precipitated material (including the VLPs) was pelleted at 14,000×g for 15 min. at 4° C. The supernatant was discarded and the pellet resuspended in 50 mM Hepes pH 7.4, 10 mM EDTA. Non-soluble debris was removed by low speed centrifugation, and the supernatant transferred to ultracentrifuge tubes for pelleting through a 30% (wt/wt) sucrose cushion. The remaining steps of the purification were identical to the steps described in the preceding paragraph.

### Example 3

### Sf21 Cell Culture

Sf21 cells are grown in TC100 medium supplemented with 10% fetal bovine serum. Addition of antibiotics (penicillin and streptomycin) is optional. The cells are maintained routinely in stationary phase and are transferred to suspension culture as needed for experiments.

### Cell Maintenance in T25 Flask

1. Resuspend cells that have formed a continuous monolayer in a T25 flask by flushing them into the medium using an automatic pipettor. Trypsin is not required as the cells do not attach very firmly to the plastic.

2. Transfer 2 ml of this cell suspension to a new T25 flask containing 8 ml fresh medium.

3. Incubate cells at 27° C. for 3-4 days and passage again.

### Cell Maintenance in T75 Flask

1. Resuspend cells that have formed a continuous monolayer in a T25 flask by flushing them into the medium using an automatic pipettor.

2. Transfer 4 ml of this cell suspension to a new T75 flask containing 20 ml fresh medium.

3. Incubate cells at 27° C. for 3-4 days and passage again.

### Growing Cells in Spinner Flasks

(Note: we use spinner flasks from Bellco. For a 200 ml culture, use a 500 ml flask to provide sufficient oxygenation during culture.)

1. Add 150 ml medium to a 500 ml spinner flask.

2. Add cells and medium from two T75 flasks to the medium; Vt˜200 ml

3. Add 2 ml of antibiotics (Stock is 100× pen/strep)

4. Place flask on a stirrer platform at 27° C. and stir at a low speed just enough to keep the cells in suspension. The cells reach a density of 1×106 cells/ml in approximately 3 days, at which point they can be used for various experiments.

### Example 4

### T. ni Cell Culture

Trichoplusia ni cells (T. ni) cells (also called High 5 cells) are only grown in suspension culture using ExCell405 medium from JRH. This is a serum-free medium. Antibiotics (pen/strep) and extra glutamine (20 mM final) is added. The cells are maintained in a 50 ml volume and expanded to 0.5-1 L as needed.

### Cell Maintenance

1. Determine cell density of the current culture.

2. Transfer 25×10⁶ cells to a new bottle (inventors use 500 ml plastic storage bottles from Corning)

3. Add medium so that the total volume is 50 ml (i.e. the cell density is 0.5×10⁶ cells)

4. Incubate at 27° C. on a shaker at approx. 100 rpm.

5. Cells will reach a density between 2-4×106 cells per ml in two days and are then passaged again.

### Expansion of Cells to 1 Liter

To grow large volumes of T. ni cells the inventors use triple baffled 2 L Fernbach Flasks (from Bellco)

1. Use cells from a 50 ml culture and transfer to a 2 L baffled Fernbach Flask

2. Add medium to approx. 250 ml total. The cell density should not be below 0.3×10⁶ per ml.

3. Incubate the cells at 27° C. on a shaker at approx. 100 rpm.

4. When the cell density reaches 2×10⁶ cells per ml (about 2 days later) add medium to 1 Liter.

5. Continue shaking the cells at 27° C. on a shaker at approx. 100 rpm.

6. When the cell density has reached 2×10⁶ cells per ml (about 2 days later), they are ready for infection with a recombinant baculovirus.

### Example 5

### Baculovirus Protocols

### Plaque Assay

The plaque assay is done with Sf21 cells.

1. Determine the density of the Sf21 cell suspension in the spinner flask.

2. Dilute the cell stock to 0.3×106 cells/ml with TC100 medium.

3. Add 5 ml of the cell suspension (1.5×106 cells total) to 60 mm tissue culture dishes. Move the plates to a separate place at room temperature and let the cells attach to the dish for 30-60 min.

4. In the meantime, prepare dilutions of the virus stock to be titered. To this end, pipet 1.8 ml medium into a series of tubes and add 0.2 ml of virus stock to the first tube. This is a tenfold dilution. Vortex this tube and transfer 0.2 ml of the tenfold dilution to the next tube and so on until a series of tubes having dilution ranging from 10-1 to 10-7 have been prepared. Typically a virus stock should have a titer of 107-108, so the inventors use only the dilutions from 10-6 to 10-8 in the assay.

5. After the cells have attached, carefully remove the medium. Add 0.5 ml of the various dilutions to the plates in a dropwise fashion. Use 3 plates per dilution.

6. Rock the plates slowly on a rocker platform for 1 hr at room temperature.

7. Remove the virus and add 5 ml of 0.5% agarose overlay (see below). Let the agarose run down the side of the dish so that the cells are not disturbed.

8. Incubate at 27° C. for 6 days.

9. Stain plates with 3 ml of 0.5% agarose containing neutral red (see below).

10. If necessary plaques can be picked (i.e. virus in the plaques can be isolated) at this point. To this end, take a sterile Pasteur pipet and attach a small bulb to its end. Place the tip of the pipet directly onto a plaque and apply gentle suction. The agarose plug and virus will be sucked into the pipet and is then transferred into a tube containing 1 ml TC100 medium by flushing up and down.

### Preparation of the Agarose Overlay:

As an example, if there are 10 plates in the assay, you will need 50 ml overlay. Make a little bit more, e.g. 60 ml. Weigh out 0.3 g low melting point agarose (we use SeaPlaque agarose) and add 60 ml medium in a sterile flask. Place the flask in a 65° C. waterbath for about 10 min. to melt the agarose. Swirl the flask occasionally. After the agarose is melted, place the flask in a 37° C. waterbath to cool everything down. Do not add the hot agarose to the cells! Add 5 ml to each plate without making bubbles and let it solidify for a few minutes.

### Neutral Red Staining:

3 ml agarose per plate is needed, i.e. 33 ml for 10 plates. Make a little bit more, e.g. 40 ml. Add 0.2 g SeaPlaque agarose to 39.3 ml sterile water. Microwave to melt the agarose. Cool down to 37° C. Add 0.72 ml of neutral red stock at 3.3 mg/ml (available as a sterile solution from SIGMA) and swirl. Add 3 ml to the center of each plate and let solidify. Incubate plates overnight at 27° C. Plaques will be visible the next day.

Propagation of Virus from a Single Plaque

1. Transfer 2.5×10⁶ Sf21 cells to a 100 mm tissue culture dish in approximately 10 ml of medium.

2. Let the cells attach for about 30-60 min.

3. Remove the medium and add the entire 1 ml of virus from the plaque pick.

4. Rock on a rocker platform at low speed for 1 hr.

5. Add 5-6 ml of fresh TC100 medium

6. Incubate at 27° C. for 5 days.

7. Harvest the medium containing the progeny virus

8. Pellet cell debris and transfer supernatant to a fresh tube. This is called PASS1 virus stock.

9. Titer the stock by plaque assay.

### Preparation of Virus Inoculum for Infection of a 1 L T. ni Cell Culture to Make VLPs

1. Determine density of Sf21 cells

2. Transfer 15×10⁶ cells to a T175 flask in 20-30 ml of medium

3. Let the cells attach for 30-60 min.

4. Remove the medium and add 15 ml fresh medium

5. Add 0.25 ml of a recombinant baculovirus stock with a titer between 10⁷-10⁸

6. Incubate the flask at 27° C. for 1 hr

7. Add another 15 ml of medium (Vt now 30 ml) and continue incubation at 27° C. until the cells have lysed (about 6 days)

8. Transfer the medium to a fresh tube and remove cell debris by low speed centrifugation

9. Transfer supernatant to fresh tube and store at 4° C.

### Infection of T. ni Cells for the Synthesis of VLPs

1. Prepare 1 L of T. ni cells at 2×10⁶ cells/ml

2. Add 30 ml of recombinant baculovirus stock prepared as described above.

3. Place the culture on a shaker at 100 rpm and incubate at 27° C. for 3-4 days.

### Example 6

### Purification of VLPs

If the cells are still intact after 3-4 days most of the VLPs will be cell associated. Proceed as follows:

1. Pellet the cells by low speed centrifugation and discard the supernatant.

2. Resuspend the cells in 200 ml 50 mM Hepes pH 7.4 (Tris buffer would be fine, too. I am not sure how important the pH is. We have used both pH 8 and pH 7.4. Both work fine.)

3. Lyse the cells with NP40 using a final concentration of 0.5% (v/v). Keep on ice for 10 min with occasional swirling of the flask.

4. Pellet cell debris at 10,000 rpm for 10 min. at 4° C. in a Beckman J2-21 centrifuge (or equivalent).

5. Transfer the supernatant to 50.2 Ti rotor tubes (Beckman) and underlay with 4 ml 30% (wt/wt) sucrose cushion in 50 mM Hepes pH 7.4, 10 mM EDTA, 0.1% (wt/v) BSA.

6. Centrifuge at 45,000 rpm for 2.5 hours at 11° C. (Note: you can also pellet the VLPs using an SW28 rotor. In this case you would centrifuge at 28,000 rpm for 5 hours at 11° C.)

7. Drain the tubes and resuspend each pellet (containing the VLPs) in 1 ml 50 mM Hepes, pH 7.4, 10 mM EDTA.

8. Prepare 10-40% (wt/wt) linear sucrose gradients in 50 mM Hepes, pH 7.4, 10 mM EDTA.

9. Apply clarified, resuspended pellet and centrifuge as follows:

SW28 rotor: 28,000 rpm, 3 hours, 11° C.

SW41 rotor: 40,000 rpm, 1.5 hours, 11° C.

Fractionate the gradient or pull off the virus band by piercing the tube with a needle and drawing the sample into a syringe.

If the cells are lysed after 3-4 days the VLPs will have been released into the medium. Proceed as follows:

1. Add NP40 to the entire 1 L cell culture using a final concentration of 0.5% (v/v)

2. Keep on ice for 10 min. with occasional swirling.

3. Pellet the cell debris by centrifugation

4. Transfer the supernatant to a large flask, add a stir bar and add NaCl to a final concentration of 0.2 M and PEG8000 to a final concentration of 8% (wt/v)

5. Stir the supernatant at 4° C. for 1 hr. The VLPs and other large molecules will precipitate.

6. Pellet the precipitate and discard the supernatant.

7. Resuspend and clarify the precipitate in 50 mM Hepes pH 7.4, 10 mM EDTA.

8. Transfer the clarified precipitate to ultracentrifuge tubes and proceed with step 5. above.

It is possible to further purify the VLPs by banding on CsCl. If so, the fractions from the sucrose gradients containing the VLPs are combined and dialysed out the sucrose. The sample is transferred to ultracentrifuge tubes and add CsCl to a final concentration of 32% (wt/wt). Centrifuge for about 18 hours at appropriate speed depending on rotor. The VLPs will form a band near the bottom of the tube. Remove the band by needle puncture.

### Example 7

### Immunisation of Fish Using MGNNV-VLPs as Vaccine Against VNN

MGNNV-VLPs were produced as described in previous examples. Vaccination of fish using MGNNV-VLPs is described thereafter.

### Vaccination of Fish.

200 sea bass Dicentrarchus labrax, average weight of 66 g each, were distributed in 8 different aquaria (25 fish each) containing 40 liters of seawater warmed at 25° C. The fish did not have food for 24 hours before vaccination. Just prior to vaccine delivery they were anesthetized using 0.2‰ (v/v) phenoxy ethanol. Two different doses of vaccine were used in triplicates: 20 μg (A1, A2, A3) and 100 μg (B1, B2, B3) of VLPs diluted in 100 μl Phosphate Buffer Saline (PBS). Fish were vaccinated by intramuscular injection. Control fish (unvaccinated) received 100 μl of PBS (A5 and B5). The mortality was recorded for 27 days after vaccination to check if the vaccine had adverse effects. 27 days after vaccine delivery, 5 fish from each aquarium were sacrificed and blood samples were taken to assay their level of plasmatic nodavirus specific antibodies by ELISA and seroneutralisation tests.

### Challenge Using a Pathogenic Fish Nodavirus Strain

On the same day (27 days post immunisation), all remaining fish (20 per aquaria) were anesthetized and injected intramuscularly with 10⁵ TCID₅₀ of nodavirus (strain W80) grown on the SSN-1 cell line. W80 is a nodavirus strain isolated in France from diseased sea bass. Previous work from inventor's laboratory has shown that it belongs to the RGNNV (red-spotted grouper nervous necrosis virus) genotype as MGNNV does. This strain is pathogenic to sea bass at 25° C. The fish behavior, the clinical signs, and the mortalities were recorded for one month after challenge. Some dead fish were kept at −80° C. for virus detection by RT-PCR. At the end of the experiment all surviving fish were sacrificed and frozen.

Serological Assays. ELISA and Seroneutralisation.

The titers of specific anti-nodavirus antibodies contained in blood samples taken 27 days after vaccination were obtained, using a sandwich ELISA method. Serial plasma dilutions were tested in order to obtain a titration curve. The antigen-antibody reaction was revealed using a colorimetric method by reading the optical density at 492 nm. The titers were expressed as the OD at 492 nm for the 1/8192 dilution. The titers of nodavirus neutralizing antibodies were obtained on the same samples than for the ELISA test. Different dilutions of the plasma from the plasma samples were incubated for 24 hours at 4° C. with W80. Then the mixtures were cultivated on the SNN-1 cell line. The neutralizing titer is expressed as the reciprocal value of the plasma dilution that gives at least 50% reduction of the titer compared of nodavirus strain W80 grown on the SSN-1 cell line (0: plasma dilution <40).

### Detection of Nodavirus by RT-PCR.

At the end of the experiment, surviving fish from the control and vaccinated groups were dissected and total RNA was extracted from their brain and their eyes. RNA from a few fish that died during the experiment was also extracted. The nodavirus nucleic acids were detected by using RT-PCR (Thiéry et al, 1999, J Fish Dis, 22, 201-208).

### Results

### 1/ Adverse Effects of the Vaccine.

The behavior of the fish (swimming, appetite, excitability) was normal in all groups. Only one fish died in one of the aquaria where the fish had received 100 μg, but it did not display any clinical signs before dying. No other mortality or morbidity was observed during the immunization step before the challenge.

2/ Mortality and Clinical Signs after Challenge.

In the unvaccinated control groups, the mortality and the typical clinical signs of VER appeared on day 5 or 6 post-infection. In one of the control groups, the cumulated mortalities reached 90% on day 9 post-infection and did not increased afterwards. In the other control group, cumulative mortalities reached 70% after 21 days post infection. The surviving fish from this group had typical clinical signs of VER (hyper-inflation of the swimming bladder, uncoordinated swimming)

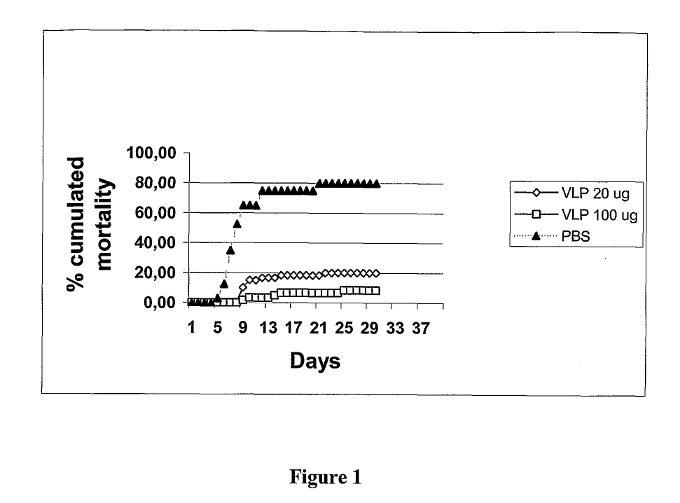

The observed mortalities in vaccinated fish were drastically lower than for unvaccinated fish. The cumulative mortalities in aquaria A1, A2 and A3 (fish vaccinated with 20 μg VLPs) at the end of the experiment were respectively of 15%, 10% and 35%. There were even lower mortalities in aquaria B1 (0%), B2 (10%) and B3 (15%), where the fish were vaccinated with 100 μg of VLPs. The average cumulated mortalities during the time course of the experiment are shown on FIG. 1.

### 3/ Serological Results.

ELISA:

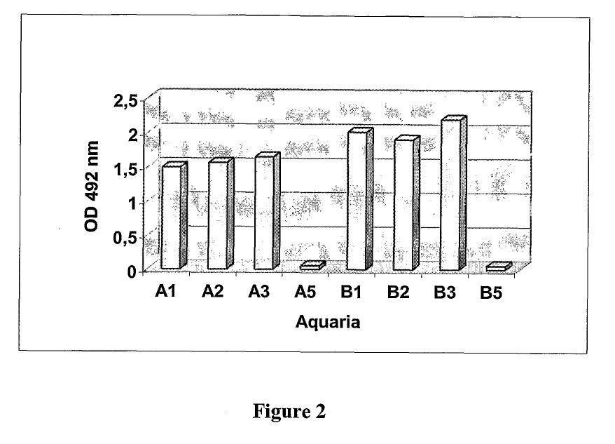

The specific anti-nodavirus antibody titers, measured in 5 fish per aquaria sampled 27 days after vaccination, are very high (OD 492 nm comprised between 1.5 and 2) in all vaccinated fish. These values are comparable to that obtained in fish naturally infected by a nodavirus. All unvaccinated fish (controls) were seronegative. The average titers obtained in the plasma of the fish from the same groups are indicated on FIG. 2.

Seroneutralisation:

The titers of nodavirus neutralizing antibodies in vaccinated fish were found to be comprised between 1280 and >5120, except for fish from one aquarium (A2) that had no neutralizing antibodies. None of the plasma from the unvaccinated fish had neutralizing antibodies.

### 4/ Detection of Nodavirus by RT-PCR

The number of surviving fish detected positive for nodavirus by RT-PCR at the end of the experiment is indicated on Table I:

TABLE I

| | Aquaria | | | | | | | |
|---|---|---|---|---|---|---|---|---|
| | | | | | | | A5 | B5 |
| | A1 | A2 | A3 | B1 | B2 | B3 | No | No |
| | 20 μg | 20 μg | 20 μg | 100 μg | 100 μg | 100 μg | vaccine | vaccine |
| +ve/total | 4/15 | 6/17 | 2/12 | 0/19 | 2/18 | 0/17 | 1/2 | 4/7 |
| % +ve | 26.6% | 35.3% | 16.6% | 0% | 11.1% | 0% | 50% | 57.1% |

The % of surviving fish that were positive for nodavirus by RT-PCR was lower for vaccinated fish than for unvaccinated fish. In the case of fish that were vaccinated with 100 μg of VLP, all surviving fish were apparently free of nodavirus in 2 aquaria out of 3, nevertheless RT-PCR performed on died fish were positive (not shown).

It is concluded from this example that MGNNV-VLPs could induce a strong immune response in vaccinated fish that confer a very high specific protection against VNN.

### Example 8

### Immunisation of Fish Using SB2-VLPs as Vaccine Against VNN

The immunogenic preparation containing purified SB2-VLPs was prepared according to previous examples. The protective potential of this preparation was tested again in sea bass following a protocol similar to the example 7. The only differences were as described thereafter.

The size of the fish was 22 g in average just prior vaccination. Several vaccine doses were tested to study the effect of the amount of VLPs upon the serological response of the fish and the extent of protection against VNN. Each vaccine dose was tested in triplicate (25 fish per replicate). The vaccine doses per fish were 20 μg, 10 μg, 5 μg, 1 μg, 0.5 μg, or 0.1 μg of SB2-VLPs. The vaccine was also delivered by intramuscular injection in 100 μl of PBS. Unvaccinated control consisted in three replicates of 25 fish receiving 100 μl of PBS.

Mortality was evaluated during 29 days after injection to assess side effects of the vaccine.

At day 29, 5 fish in each recipient are sacrificed so as to search for anti-nodavirus plasmatic antibodies.

At day 30, every remaining fish were given intramuscular injection of 100 μl of SSN-1 cellular culture supernatant containing 10⁵ TCID₅₀ of strain W80, which is virulent at 25° C.

The behaviour of the fish as well as clinical signs and mortality were determined daily during 29 days. Dead fish were conserved to search for nodavirus by RT-PCR.

### Results

### Search for Side Effects

The behaviour of the fish (swimming, appetite and excitability) appeared normal in all cases. Two fish were dead without any specific explanation. No other mortality or morbidity was observed during the test phase.

Mortality after Viral Challenge

In the negative controls (no vaccination), mortality and clinical signs appeared at day 5. Mean cumulated mortality at the end of the test, i.e. 29 days after viral challenge, was 46.7%.

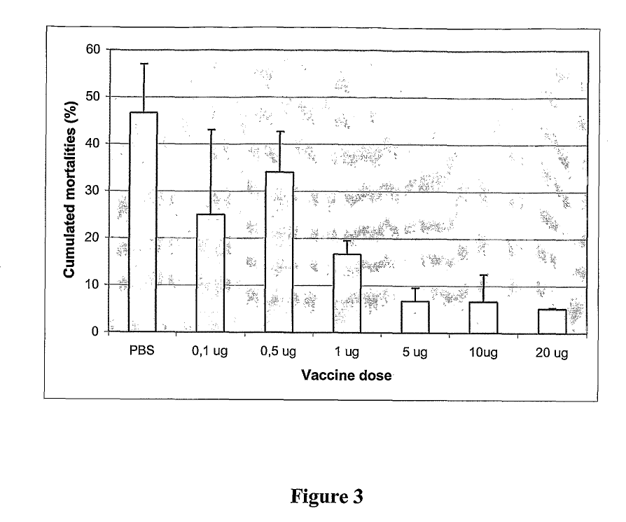

Basically, cumulated mortality observed at day 29 in a VLP injected fish is lower when the received VLP dose was increased: 25% (0.1 μg); 33.9% (0.5 μg); 16.7% (1 μg); 6.8% (5 μg); 6.7% (10 μg); 5.2% (20 μg). As a consequence, the inventors concluded that there is a correlation between the vaccine dose and the protection conferred (see FIG. 3).

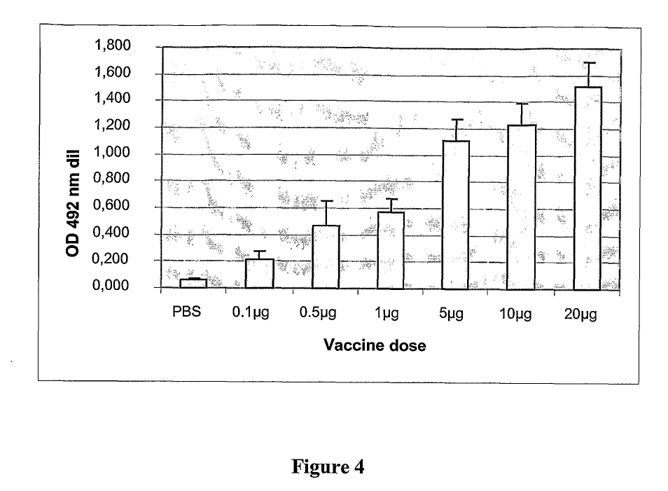

Mean titer of anti-nodavirus plasmatic antibodies, expressed as the OD at 492 nm obtained by ELISA for plasma dilution 1/8192, are presented in FIG. 4. The results showed that the anti-nodavirus antibody titer is positively correlated with the dose of administered vaccine. Even at very low dose (0.1 μg) the titer is significantly above the titer measured in negative control fish.

Seroneutralisation

Neutralizing antibodies titers were measured and the results are presented in table II below

TABLE II

| | Amount of vaccine (μg/fish) | | | | | | |
|---|---|---|---|---|---|---|---|
| | No | | | | | | |
| | vaccine | 0.1 μg | 0.5 μg | 1 μg | 5 μg | 10 μg | 20 μg |
| Titers | <40 | <40- | <40- | 1280- | 1280- | 1280- | 1280- |
| Neutralizing | | 320 | 1280 | 2560 | 2560 | 5120 | >5120 |
| Antibodies | | | | | | | |
| (min-max) | | | | | | | |

These results demonstrated that the titer of antibody capable of neutralizing the nodavirus increases with the vaccine dose administered to the fish. The inventors have therefore drawn the conclusion that the vaccine has the ability to protect the fish against nodavirus infection but is also able to induce the synthesis of antibodies capable of neutralizing the virus in vaccinated fish. This explains also the fact that the mortality rate in vaccinated fish was lower in fish groups presenting the higher titers of specific and neutralizing antibodies.

### Detection of Nodavirus by RT-PCR.

In order to verify that the virus is indeed present in the infected fish, the inventors searched for the viral genome by RT-PCR in dead fish in the course of the test as well as in survivor fish as the end of the experiment or in survivor fish presenting clinical sign of VNN. The results are presented in table III.

TABLE III
Number of positive fish/Number of tested fish

| | | | Survivors at the end of |
|---|---|---|---|
| | | | the experiment |
| | | | (apparently healthy or |
| Fish group (μg/fish) | | Dead fish | with clinical signs) |
| Controls | | 19/19 | 24/24 |
| 0.1 | μg | 14/14 | 17/18 |
| 0.5 | μg | 18/20 | 16/16 |
| 1 | μg | 9/10 | 16/17 |
| 5 | μg | 4/4 | 14/15 |
| 10 | μg | 4/4 | 13/15 |
| 20 | μg | 3/3 | 6/16 |

The PCR test detected the nodavirus genome in most of the dead fish except three. On the contrary, the percentage of survivor fish at the end of the experiment in which the viral genome is no more detectable by RT-PCR is more important in vaccinated fish with VLP according to the invention at 20 μg.

### CONCLUSION

The results of these experiment have confirmed that the SB2-VLP preparation according to the invention is capable of inducing an efficient protection against a nodavirus infection in sea bass. Results have demonstrated that mortality after challenge is decreasing when the vaccine dose is increasing and that the titer of neutralizing anti-nodavirus antibodies is higher when the vaccine dose is increased.

### Example 9

### Immunisation of Fish Using SB1-VLPs Administered by Intramuscular or by Intraperitoneal Injection

### Material and Methods

SB1-VLPs were prepared according to previous examples. 300 juvenile sea bass (average weight 2.5 g) were held in aerated sea water in 12 different aquaria (25 fish per aquaria). Fish were vaccinated according to previous examples using 5 μg of SB1-VLPs per fish. Two modes of administration i.e. intramuscular (IM) or intraperitoneal (IP) injection were tested in fish held at 15° C. or 20° C. Each treatment was tested in duplicate.

Unvaccinated controls consisted in:
IP or IM injection of 1001 of PBS buffer in fish held at 15° C.
IP or IM injection of 1001 of PBS buffer in fish held at 20° C.

After 19 days post-vaccination, 5 fish in each aquaria were sacrificed and blood samples were taken for antinodavirus antibody testing (ELISA).

### Results.

### Specific Antinodavirus Antibodies in Vaccinated Fish.

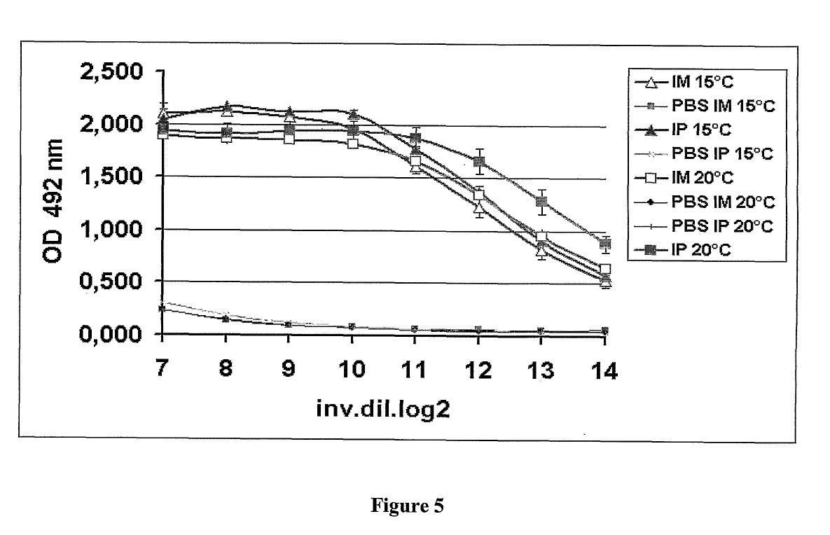

The level of plasmatic antinodavirus antibodies is shown on FIG. 5. High level of specific antibodies were detected in all vaccinated fish whatever the administration mode of the vaccine. Unvaccinated controls were seronegative. Interestingly, the titer of antinodavirus antibody was slightly higher when fish were vaccinated at 20° C. by IP injection compared to the other group of fish. This could reflect an better immune response of this fish species at 20° C. compared to 15° C. Alternatively, the volume of vaccine delivered by IP injection could be slightly higher than by IM injection.

This example demonstrates that SB1-VLPs could also induce a strong immune response to juvenile sea bass fish in our experimental conditions. In this experiment, the serological status of the fish was tested 19 days after vaccination. The titer of the specific antibodies was found to be in the same order of magnitude of previous examples (i.e. when fish were tested after about. one month after vaccination). Thus, it is likely that the specific immune response induced by nodavirus VLPs could also protect fish against a nodaviral challenge rather shortly after vaccination.

### Example 10

### Immunisation of Fish Using SB2-VLPs Administered by Bath and by Intraperitoneal Injection

### Material and Methods.

In order to test the protective effect against VNN when using other vaccine administration methods (bath and intraperitoneal vaccination), another experiment was set using SB2-VLPs prepared according to the previous examples. In addition, a partially purified (pp) preparation of SB2-VLPs was also tested. This vaccine preparation was prepared as described in the previous examples except that the VLPs contained in the lysate of Tni cells infected by the recombinant SB2 baculovirus were concentrated by ultracentrifugation through a sucrose cushion. An uninfected Tni cell lysate was prepared and treated in the same way for control purpose. No further ultracentrifugation step was involved (i.e. no CsCl gradient purification step as in previous examples). For the bath exposure method, two doses of SB2-VLPs were used (approx. 5 μg or 50 μg/per fish). Each vaccine preparation was tested in duplicate using 40 sea bass juveniles weighing 4.5 g (average weight) held in sea water warmed at 25° C.±1° C. Fish were exposed to the vaccine added in the bath for 1 hour in 1 litre of aerated sea water. Fish vaccinated by intraperitoneal injection received 5 μg of purified or partially purified SB2-VLPs per fish in 100 μl of PBS.

Unvaccinated controls also consisted in duplicate trials (40 fish each):
Intraperitoneal injection of 100 μl of PBS buffer
Intraperitoneal injection of 100 μl of uninfected—Tni cells lysate (no VLPs).
Bath exposure to sea water containing uninfected—Tni cells lysate (no VLPs) for one hour.

After 29 days post-vaccination, 5 fish in each aquaria were sacrificed and blood samples were taken for antinodavirus antibody testing (ELISA).

At 30 days post-vaccination all fish were challenged by intramuscular injection (strain W80, 9×10⁶ TCID₅₀/per fish).

### Results.

Mortality and Clinical Signs after Challenge.

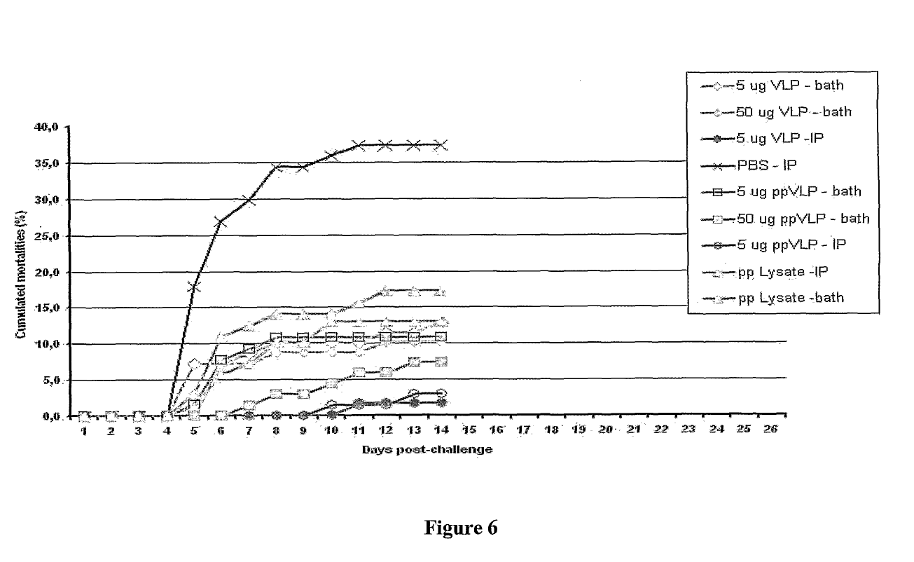

Cumulated mortality after challenge is represented on FIG. 6. Some fish started to display clinical signs of VNN between 4 and 6 days post-challenge in most of the aquaria except when fish were vaccinated by intraperitioneal injection. Heavy mortality appeared at day 5 in the unvaccinated control (10 fish out of 35 died in one aquaria). In other groups the onset of mortality was delayed. The best protection is observed when the fish are vaccinated by intraperitoneal injection either by the purified or the partially purified VLPs preparation. Bath exposure to the higher dose of VLPs used in this experiment appears to decrease significantly the mortality, particularly using the partially purified preparation. Interestingly, the cell lysate alone appears to induce some protection, whatever the administration method, probably through an unspecific protection mechanism. Fourteen days after challenge a significant proportion of fish displaying clinical signs is present in all groups except the intraperitoneally vaccinated groups. Nevertheless, the number of apparently infected fish is lower in fish vaccinated by bath using 50 μg of VLPs per fish.

### Specific Antinodavirus Antibodies in Vaccinated Fish.

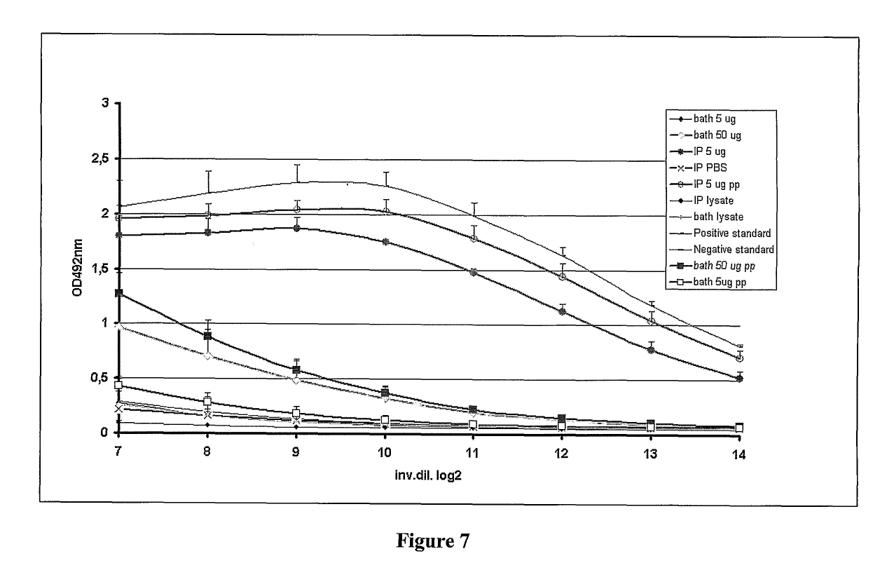

The level of plasmatic antinodavirus antibodies is shown on FIG. 7. The higher level of antibodies were observed in fish vaccinated by intraperitoneal injection, which is in agreement with the observed protection against challenge. Bath exposure to purified or partially purified (pp) VLPs at 50 μg per fish also elicited a specific immune response. However, the antinodavirus antibody titers are lower than for intraperitoneally vaccinated fish. Nevertheless, a significant increase of the OD at 492 nm compared to the negative controls is still observed for plasma dilution of 1/1024. Antibody titers measured in the fish from other groups did not differ significantly from negative controls.

This example demonstrates that intraperitoneal injection of small fish with as low of 5 μg of SB2-VLPs per fish confers strong protection against an homologous viral challenge as intramuscular injection does. Partially purified VLPs also conferred good protection against VNN by intraperitoneal injection. Bath vaccination protects against VNN compared to unvaccinated controls, but the protection is lower than using the intraperitoneal route of vaccination. Moreover, the protection was only observed when fish were exposed to a bath containing the vaccine at 50 μg of SB2-VLPs per fish. As in previous examples, the level of protection against nodaviral challenge is correlated to the titer of specific antinodavirus antibodies detected in the plasma of vaccinated fish. Thus, bath vaccination using a higher concentration of VLPs increases both the antibody levels in fish and protection against challenge. There was a significant increase of protection using the partially purified VLPs over the purified VLPs. At present it is not known if this is due to differences in the vaccine composition or if it actually reflects minor differences in the VLPs concentrations of the vaccines. On the other hand, residual Tni cell components that are present in the partially purified VLPs preparation could elicit an unspecific immune response. This is supported by the observed decrease of mortality in fish groups that were vaccinated with a partially purified preparation of mock-infected Tni cell lysates whereas those fish did not bear specific antinodavirus antibodies.

1.-28. (canceled)

29. An immunogenic composition for fish comprising nodavirus virus-like particles (VLPs) formed with nodavirus capsid protein assembly.

30. The immunogenic composition of claim 29, wherein the VLPs comprise at least one nodavirus capsid protein having an amino acid sequence selected from the group consisting of SEQ ID NO: 5, SEQ ID NO: 6, SEQ ID NO: 7, SEQ ID NO: 8 and variants thereof having at least 70% identity with any one of SEQ ID NO: 5, SEQ ID NO: 6, SEQ ID NO: 7, or SEQ ID NO: 8.

31. The immunogenic composition of claim 29, wherein the nodavirus capsid protein is encoded by a nucleic acid sequence selected from the group consisting of SEQ ID NO: 1, SEQ ID NO: 2, SEQ ID NO: 3 and SEQ ID NO: 4 and variants thereof having at least 80% identity with any one of SEQ ID NO: 1, SEQ ID NO: 2, SEQ ID NO: 3 or SEQ ID NO: 4.

32. The immunogenic composition of claim 29, wherein the VLPs are obtained by a process comprising the steps of: a) infecting host cells with a recombinant vector that expresses the nodavirus capsid protein; b) obtaining a host cell lysate comprising the nodavirus VLPs; and c) optionally extracting and purifying the VLPs assembled from the host cell lysate.

33. The immunogenic composition of claim 32, wherein the recombinant vector is a recombinant baculovirus and the host cells are insect cells.

34. The immunogenic composition of claim 33, wherein the insect cells are Sf21 cells or T. ni cells.

35. The immunogenic composition of claim 32, wherein the nodavirus capsid protein is encoded by a nucleic acid sequence selected from the group consisting of SEQ ID NO: 1, SEQ ID NO: 2, SEQ ID NO: 3 and SEQ ID NO: 4 and variants thereof having at least 80% identity with any one of SEQ ID NO: 1, SEQ ID NO: 2, SEQ ID NO: 3 or SEQ ID NO: 4.

36. The immunogenic composition of claim 29, which comprises a mixture of at least two VLPs, wherein each VLP comprises a different nodavirus capside protein.

37. The immunogenic composition of claim 29, further comprising a pharmaceutically acceptable adjuvant.

38. A vaccine comprising the immunogenic composition of claim 37 suitable for administration to fish.

39. The vaccine of claim 38, wherein the fish is Dicentrarchus labrax, Epinephelus sp. or a fish species susceptible to nodavirus infection.

40. The vaccine of claim 38, wherein said administration is via the intramuscular or the intraperitoneal route.

41. The vaccine of claim 40, wherein said administration is from about 0.5 μg to about 200 μg of VLPs per 100 g of fish.

42. The vaccine of claim 41, wherein said administration is from about 1 μg to about 20 μg of VLPs per 100 g of fish.

43. The vaccine of claim 42, wherein said administration is from about 1 μg to about 5 μg of VLPs per 100 g of fish.

44. The vaccine of claim 38, wherein said administration is by bath and/or via the oral route.

45. The vaccine of claim 44, wherein said administration is from about 0.5 μg to about 200 mg of VLPs per 100 g of fish.

46. The vaccine of claim 45, wherein said administration is from about 500 μg to about 150 mg of VLPs per 100 g of fish.

47. The vaccine of claim 46, wherein said administration is from about 1 mg to about 100 mg of VLPs per 100 g of fish.

48. A method of manufacturing a vaccine for treating or preventing a nodavirus infection in fish comprising the steps of: a) infecting host cells with a recombinant vector that expresses the nodavirus capsid protein; b) obtaining a host cell lysate comprising the nodavirus VLPs; c) optionally extracting and purifying the VLPs assembled from the host cell lysate; and d) adding a pharmaceutically acceptable adjuvant to the nodavirus VLPs to form a VLP vaccine composition.

49. The method of claim 48 wherein the recombinant vector is a recombinant baculovirus and the host cells are insect cells.

50. The method of claim 49, wherein the insect cells are Sf21 cells or T. ni cells.

51. The method of claim 48, wherein the nodavirus infection in fish is selected from the group consisting of viral encephalopathy, retinopathy and viral nervous necrosis.

52. The method of claim 48, wherein the fish are raised in fish farming and are at the larval and juvenile stages of development or are broodstock fish.

53. A fish farming bath comprising the immunogenic composition of claim 29 in an amount from about 0.5 μg to about 200 mg of VLPs per 100 g of fish.

54. A method for treating or preventing nodavirus infection in fish comprising administering a concentrate of the immunogenic composition of claim 29 to a fish farming bath in a pharmaceutically effective amount.

55. The method of claim 54, wherein said pharmaceutically effective amount is from about 0.5 μg to about 200 mg of VLPs per 100 g of fish.

56. A method of treating of preventing nodavirus infection in fish comprising the steps of a) introducing said fish in a bath; and b) adding a pharmaceutically effective amount of the immunogenic composition of claim 29 to the bath to allow stimulation of the fish immune system.

57. The method of claim 56 wherein said pharmaceutically effective amount is from about 0.5 μg to about 200 mg of VLPs per 100 g of fish.

58. A food composition for oral immunization of fish comprising a pharmaceutically effective amount of nodavirus virus-like particles (VLPs) formed with nodavirus capsid protein assembly, and a pharmaceutically acceptable adjuvant.

59. The food composition of claim 58 wherein said VLPs comprise at least one nodavirus capsid protein having an amino acid sequence selected from the group consisting of SEQ ID NO: 5, SEQ ID NO: 6, SEQ ID NO: 7, SEQ ID NO: 8 and variants thereof having at least 80% identity with any one of SEQ ID NO: 5, SEQ ID NO: 6, SEQ ID NO: 7, or SEQ ID NO: 8.

60. A method for treating or preventing nodavirus infection in fish comprising administering to a fish in need thereof the vaccine of claim 38 in a pharmaceutically effective amount.

61. The method of claim 60, wherein the nodavirus infection in fish is selected from the group consisting of viral encephalopathy, retinopathy and viral nervous necrosis.


SEQUENCE LISTING (18 sequences)

SEQ ID NO: 1 - DNA, 1017 bp, Dicentrarchus labrax encephalitis virus
  Nucleic acid sequence encoding the SB2 nodaviral capsid protein
atggtacgcaaaggtgataagaaattggcaaaacccgcgaccaccaaggccgccaatccgcaaccccgccgacgtgctaacaatcgtcggcgtagtaatcgcactgacgcacctgtgtctaaggcctcgactgtaactggatttggacgtgggaccaatgacgtccatctctcaggcatgtcgagaatctcccaggccgtcctcccagccgggacaggaacagacggatacgttgttgttgacgcaaccatcgtccccgacctcctgccacgactgggacacgctgctagaatcttccagcgatacgctgttgaaacactggagtttgaaattcagccaatgtgccccgcaaacacgggcggtggttacgttgctggcttcctgcctgatccaactgacaacgatcacaccttcgacgcgcttcaagcaactcgtggtgcagtcgttgccaaatggtgggaaagcagaacagtccgacctcagtacacccgcacgctcctctggacctcgtcgggaaaggagcagcgtctcacgtcacctggtcggctgatactcctgtgtgtcggcaacaacactgatgtggtcaacgtgtcagtgctgtgtcgctggagtgttcgattgagcgttccatctcttgagacacctgaagagactaccgctcccatcatgacacaaggttccctgtacaacgattccctttccacaaatgatttcaagtccatcctcctaggatccacaccactggacattgcccctgatggagcagtcttccagctggaccgtccgctgtccattgactacagccttggaactggagatgttgaccgtgctgtttattggcacatcaagaagtttgctggaaatgctggcacacctgcaggctggtttcgctggggcatctgggacaacttcaacaagacgttcacagatggcgttgcctactactctgatgagcagccccgtcaaatcctgctgcctgttggcactgtctgcacccgtgttgactcggaaaactaa

SEQ ID NO: 2 - DNA, 1406 bp, Dicentrarchus labrax encephalitis virus
  Nucleic acid sequence encoding the SB1 nodaviral capsid protein
ctttgcatcccacaatggtacgcaagggtgagaagaaattggctaaaacagcgaccaccaaggccgctaatccccaaccccgtcgacgtgccaccaaccggcggcgtagtaatcgacctgacgcgcctttagcaaaggcttcgactgtcacgggatttggacgtgggaccaatgacgtccatctctcgggtatgtcgagaatctcccaagcagtcctcgcggccggggcaggaaccgacggatacatcgtcgttgattcgaccatcgtccccgacatcctgccacgactgggacacgctgctagaatcttccagcgatacactgttgaaacattggagttcgaagttcagccaatgtgccccgcaaacacgggcggtggttacgttgctggcttcctgcctgatccaactgacaacgaccacaccttcgacgcgcttcaagcgactcgtggtgccgttgttggcaaatggtgggagagcaggacagtacgtccacagtacactcgaaccatgctctggacctcaacaggaaaggagcagcgtttgacttcacctggacgcttcattctcctctgtgtcggcagcaacactgacgtggttaacgtgtcggtgttatgtcgctggagtgtggccctcagcgtcccatctctcgagacgcctgaggacacagctgcacctattttgtcacagggtcccctctacaacgattcccttgccacatctgactttaaatccatcctcctgggttccacacagctggacatatccccggacggcgccatcttccagatggaccgcccgctgtccattgattacaagctgggaaccggagatgttgaccgtgccgtttattggcacctcaagaagtttgcgggcactgccaccacaccggctggctggtttcgctggggcatctgggacaacttcaacaaaacattcaccgacggtatcgcttactattctgatgagcagccacgccagatcctgctcccggtaggcactaccttcaccagggtcgatgcgggaaactaaccgggtcatcggttccctagtgcgtatcgttgatgaccaatttgaacaattaattaatgcactaagtattataactaatgaaatacaaataaacaaaactgagattggcaagaataagagcgaaattgacgctatcgccactaaattaaaagacaaagcccccaaggagggtgctattgctattgttggtaccattgacggcgtaccggctacgttgacggcctttacacgccacactgccgcgtgcctaatagggtgccagctttaccagtcgtatccacgccgaggatttccccccttgggcatgttgggttaccgtcagctccacgtaacgacccatgtggctaaatggctgatggctacccagtttcggcg

SEQ ID NO: 3 - DNA, 377 bp, Dicentrarchus labrax encephalitis virus
  Nucleic acid sequence encoding the Y325 nodaviral capsid protein
agtgtggccctcagcgtcccatctctcgagacgcctgaggacacagctgcacctattttgtcacagggtcccctctacaacgattcccttgccacatctgactttaaatccatcctcctgggttccacacagctggacatatccccggacggcgccatcttccagatggaccgcccgctgtccattgattacaagctgggaaccggagatgttgaccgtgccgtttattggcacctcaagaagtttgcgggcactgccaccacaccggctggctggtttcgctggggcatctgggacaacttcaacaaaacattcaccgacggtatcgcttactattctgatgagcagccacgccagatcctgctcccggtaggcac

SEQ ID NO: 4 - DNA, 1390 bp, Malabaricus nervous necrosis virus
  Nucleic acid sequence encoding the MGNNV nodaviral capsid protein
ctttgcaagtcaaaatggtacgcaaaggtgaaaagaaattggcaaaacccgcgaccaccaaggccgcgaatccgcaaccccgccgacgtgccaacaatcgtcggcgtagcaatcgcactgacgcacctgtgtctaaggcctcgactgtaactggatttggacgtgggaccaatgacgtccatctctcaggtatgtcgagaatctcccaggccgtcctcccagccgggacaggaacagacggatacgttgttgttgatgcaaccatcgtccccgacctcctgccacgactgggacacgctgctagaatcttccagcgatacgctgttgaagcactggagtttgaaattcagccaatgtgccccgcaaacacgggcggtggttacgttgctggcttcctgcctgatccaactgacaacgatcacaccttcgacgcgcttcaagcaactcgtggtgcagtcgttgccaaatggtgggaaagcagaacagtccgacctcagtacacccgcacgctcctctggacctcgacgggaaaggagcagcgtctcacgtcacctggtcggctgatactcctgtgtgtcggcaacaacactgatgtggtcaacgtgtcagtgctgtgtcgctggagtgttcgactgagcgttccatctcttgagacacctgaagagaccaccgctcccatcatgacacaaggttccctgtacaacgattccctttccacaaatgacttcaaatccatcctcctaggatccacaccactggacattgcccctgatggagcagtcttccagctggaccgtccgctgtccattgactacagccttggaactggagatgttgaccgtgctgtttactggcacctcaagaagtttgctggaaatgctggcacacctgcaggctggtttcgctggggcatctgggacaacttcaacaagacgttcacagatggcgttgcctactactctgatgagcagccccgtcaaatcctgctgcctgttggcactgtctgcaccagggttgactcggaaaactaaccgggtcatccggttccctagtgcgtatcgttgatgaccaatttgaacaattgattaaagcactaacaaatataaataaagaaatacaaacaaacaaaactgaaattggaaagaatagaagcgaaattgaatcactcgctagcaaattaaatgacaaagcacccaaggagggtgcgattgctattgttggtacccttgacggcgtaccggctacgcttgaaggcctgtacacggctggaagcgcgccgcgtgcttaattgggtgccagtggtaccagtcgtatccaacgccgaggaagtccctcgttgggctgttgggttaccgttagctccgcgcagtgagcaccaccgccatgtggt

SEQ ID NO: 5 - protein, 338 aa, Dicentrarchus labrax encephalitis virus
  Amino-acid sequence of the SB2 nodaviral capsid protein
MVRKGDKKLAKPATTKAANPQPRRRANNRRRSNRTDAPVSKASTVTGFGRGTNDVHLSGMSRISQAVLPAGTGTDGYVVVDATIVPDLLPRLGHAARIFQRYAVETLEFEIQPMCPANTGGGYVAGFLPDPTDNDHTFDALQATRGAVVAKWWESRTVRPQYTRTLLWTSSGKEQRLTSPGRLILLCVGNNTDVVNVSVLCRWSVRLSVPSLETPEETTAPIMTQGSLYNDSLSTNDFKSILLGSTPLDIAPDGAVFQLDRPLSIDYSLGTGDVDRAVYWHIKKFAGNAGTPAGWFRWGIWDNFNKTFTDGVAYYSDEQPRQILLPVGTVCTRVDSEN

SEQ ID NO: 6 - protein, 338 aa, Dicentrarchus labrax encephalitis virus
  Amino-acid sequence of the SB1 nodaviral capsid protein
MVRKGEKKLAKTATTKAANPQPRRRATNRRRSNRPDAPLAKASTVTGFGRGTNDVHLSGMSRISQAVLAAGAGTDGYIVVDSTIVPDILPRLGHAARIFQRYTVETLEFEVQPMCPANTGGGYVAGFLPDPTDNDHTFDALQATRGAVVGKWWESRTVRPQYTRTMLWTSTGKEQRLTSPGRFILLCVGSNTDVVNVSVLCRWSVALSVPSLETPEDTAAPILSQGPLYNDSLATSDFKSILLGSTQLDISPDGAIFQMDRPLSIDYKLGTGDVDRAVYWHLKKFAGTATTPAGWFRWGIWDNFNKTFTDGIAYYSDEQPRQILLPVGTTFTRVDAGN

SEQ ID NO: 7 - protein, 126 aa, Dicentrarchus labrax encephalitis virus
  Amino-acid sequence of the Y325 nodaviral capsid protein
SVALSVPSLETPEDTAAPILSQGPLYNDSLATSDFKSILLGSTQLDISPDGAIFQMDRPLSIDYKLGTGDVDRAVYWHLKKFAGTATTPAGWFRWGIWDNFNKTFTDGIAYYSDEQPRQILLPVGT

SEQ ID NO: 8 - protein, 338 aa, Malabaricus nervous necrosis virus
  Amino-acid sequence of the MGNNV nodaviral capsid protein
MVRKGEKKLAKPATTKAANPQPRRRANNRRRSNRTDAPVSKASTVTGFGRGTNDVHLSGMSRISQAVLPAGTGTDGYVVVDATIVPDLLPRLGHAARIFQRYAVEALEFEIQPMCPANTGGGYVAGFLPDPTDNDHTFDALQATRGAVVAKWWESRTVRPQYTRTLLWTSTGKEQRLTSPGRLILLCVGNNTDVVNVSVLCRWSVRLSVPSLETPEETTAPIMTQGSLYNDSLSTNDFKSILLGSTPLDIAPDGAVFQLDRPLSIDYSLGTGDVDRAVYWHLKKFAGNAGTPAGWFRWGIWDNFNKTFTDGVAYYSDEQPRQILLPVGTVCTRVDSEN

SEQ ID NO: 9 - DNA, 27 bp, Artificial sequence
  Primer SB2 N-term
accagatctatggtacgcaaaggtgat

SEQ ID NO: 10 - DNA, 29 bp, Artificial sequence
  Primer SB2 C-term
taagcggccgcttagttttccgagtcaac

SEQ ID NO: 11 - DNA, 27 bp, Artificial sequence
  Primer SB1 N-term
accagatctatggtacgcaagggtgag

SEQ ID NO: 12 - DNA, 29 bp, Artificial sequence
  Primer SB1 C-term
taagcggccgcttagtttcccgcatcgac

SEQ ID NO: 13 - DNA, 21 bp, Artificial sequence
  Primer Bac1
accatctcgcaaataaataag

SEQ ID NO: 14 - DNA, 20 bp, Artificial sequence
  Primer Bac2
acaacgcacagaatctagcg

SEQ ID NO: 15 - DNA, 21 bp, Artificial sequence
  Primer N1
cgctttgcaagtcaaaatggt

SEQ ID NO: 16 - DNA, 21 bp, Artificial sequence
  Primer N2
accacatggcggtggtgctca

SEQ ID NO: 17 - DNA, 21 bp, Artificial sequence
  Primer NW1
cgctttggaattcaaaatggt

SEQ ID NO: 18 - DNA, 18 bp, Artificial sequence
  Primer NW2
tttatctagatggcggtg